(19) United States
(12) Patent Application Publication
(10) Pub. No.: US 2009/0088803 A1
(43) Pub. Date: Apr. 2, 2009

(54) FLEXIBLE MEMBERS FOR CORRECTING SPINAL DEFORMITIES

(75) Inventors: Jeff Justis, Germantown, TN (US); Hai H. Trieu, Cordova, TN (US)

Correspondence Address: MEDTRONIC;Attn: Noreen Johnson - IP Legal Department, 2600 Sofamor Danek Drive, MEMPHIS, TN, 38132, US

(73) Assignee: WARSAW ORTHOPEDIC, INC., Warsaw, IN (US)

(21) Appl. No.: 11/865,326

(22) Filed: Oct. 1, 2007

(51) Int. Cl. A61B 17/70; A61B 17/56; A61B 17/58; A61B 17/04; A61B 19/00

(52) U.S. Cl. 606/254; 606/103; 128/898; 606/301; 606/264; 606/246; 606/308

(57) ABSTRACT

The present application is directed to devices and methods for correcting a spinal deformity. The devices may include a member attached to one or more vertebral members of a deformed spine. The member may be constructed of a flexible material with elastic properties. The member is attached to the vertebral members in a stressed orientation. Due to the elastic properties of the material, the member exerts a corrective force on the vertebral members. In some embodiments, multiple members are attached to the vertebral members to apply the corrective force.

### BACKGROUND

The present application is directed to devices and methods for correcting a spinal deformity, and more particularly, to flexible corrective members that are attached to the vertebral members to apply a corrective force to treat the spinal deformity.

The normal spine possesses some degree of curvature in three different regions. The lumbar spine is normally lordotic (that is, concave posteriorally), the thoracic spine kyphotic (i.e. convex posteriorally), and the cervical spine is also lordotic. These curvatures are necessary for normal physiologic function, and correction is desirable when the spine has either too much or too little curvature in these regions as compared with the norm. A more common abnormality, however, is lateral deviation of the spine or scoliosis.

The first successful internal fixation method for surgically treating scoliosis involves the use of the Harrington instrumentation system. In this method, a rigid member having hooks is implanted adjacent the concave side of the scoliotic spine. The hooks engage in the facet joints of a vertebral member above and under the lamina of the vertebral member below the abnormally curved region. At the time of surgery, the spine is manually straightened to a desired extent. A distraction member is then used to maintain the correction by exerting vertical forces at each end on the two aforementioned vertebral members. The member commonly has a ratcheted end over which the hooks are slidably mounted and locked in place. The effective length of the member may thus be adjusted to an appropriate length for exerting the distractive force.

The Harrington distraction member, because its corrective force is purely distractive, tends to correct curvature in both the frontal and sagittal planes. This means that unwanted loss of normal thoracic kyphosis or lumbar lordosis may inadvertently be produced. To compensate for this, a compression member is sometimes placed on the convex side of the scoliotic spine. Another variation on the Harrington method which addresses the same problem is to contour the distraction member in a sagittal plane in accordance with the kyphotic and lordotic curvatures of the normal spine. This may, however, reduce the ability to apply large corrective forces in the frontal plane due to column buckling.

The Harrington instrumentation system has been used successfully but exhibits some major problems. It requires a long post-operative of external immobilization using a cast or brace. Also, because the distraction member is fixed to the spine in only two places, failure at either of these two points means that the entire system fails. Failure at the bone-hook interface is usually secondary to mechanical failure of the bone due to excess distractive force.

Another problem with the aforementioned Harrington instrumentation system is its lack of effectiveness in producing rotary correction in the transverse plane. The longitudinal forces of the Harrington distraction method, with or without an additional compression member, do not contribute a corrective force necessary for transverse plane de-rotation. This is unfortunate because scoliosis is generally a three-dimensional deformity requiring some correction in the transverse plane.

### SUMMARY

The present application is directed to devices and methods for correcting a spinal deformity. The devices may include a member attached to one or more vertebral members of a deformed spine. The member may be constructed of a flexible material with elastic properties. The member is attached to the vertebral members in a stressed orientation. Due to the elastic properties of the material, the member exerts a corrective force on the vertebral members. In some embodiments, multiple members are attached to the vertebral members to apply the corrective force.

### BRIEF DESCRIPTION OF THE DRAWINGS

FIG. 1 is a schematic coronal view of an example of a scoliotic spine.

FIG. 2 is a perspective view of a member according to one embodiment.

FIG. 3 is a schematic view of a member according to one embodiment.

FIG. 4 is a sectional view in the axial plane of an anchor attached to a vertebral member according to one embodiment.

FIG. 5 is a schematic view of anchors attached to the vertebral members along a section of the spine according to one embodiment.

FIG. 6 is a perspective view of an extender attached to an anchor according to one embodiment.

FIG. 7 is a perspective view of a member and an inserter according to one embodiment.

FIG. 8 is a perspective view of a member being inserted percutaneously into a patient according to one embodiment.

FIG. 9 is a schematic view of a member attached to anchors along a section of the spine according to one embodiment.

FIG. 10A is a schematic view of a deformed spine and a pre-bent member according to one embodiment.

FIG. 10B is a schematic view of a deformed spine with a pre-bent member attached to anchors along a section of the spine according to one embodiment.

FIG. 10C is a schematic view of a deformed spine with a pre-bent member attached to anchors along a section of the spine according to one embodiment.

FIG. 11 is an exemplary stress-strain diagram according to one embodiment.

FIG. 12 is a perspective view of a member according to one embodiment.

FIG. 13 is a schematic view of members and anchors attached to the vertebral members along a section of the spine according to one embodiment.

FIG. 14 is a schematic view of a member attached to anchors along a section of the spine according to one embodiment.

FIG. 15 is a schematic view of a pair of members attached to anchors along a section of the spine according to one embodiment.

### DETAILED DESCRIPTION

The present application is directed to devices and methods for correcting a spinal deformity. One embodiment includes a flexible member that assumes a neutral, non-stressed orientation when not under the influence of external forces. The member is deformed to a second, stressed orientation and attached to vertebral members along the spinal deformity. The flexible member desires to return towards the neutral, non-stressed orientation and thus applies a corrective force to the vertebral members to treat the spinal deformity. The flexible member may be placed in the stressed orientation and attached to the vertebral members by a variety of different methods. Multiple members may be attached to the vertebral members to treat the various aspects of the spinal deformity.

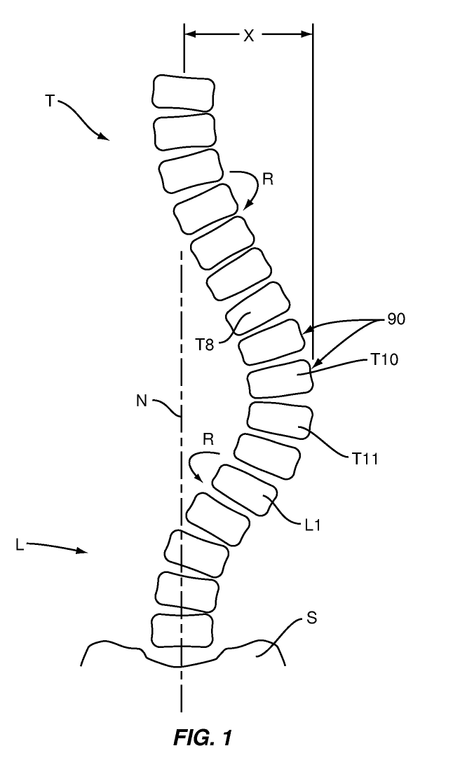

FIG. 1 illustrates a patient's spine that includes a portion of the thoracic region T, the lumbar region L, and the sacrum S. This spine has a scoliotic curve with an apex of the curve being offset a distance X from its correct alignment N in the coronal plane. The spine is deformed laterally and rotationally so that the axes of the vertebral members 90 are displaced from the sagittal plane passing through a centerline of the patient. In the area of the lateral deformity, each of the vertebral members 90 includes a concave side and a convex side. One or more of the vertebral members 90 may be further misaligned due to rotation as depicted by the arrows R. As a result, the axes of the vertebral members 90 which are normally aligned along the coronal plane are non-coplanar and extend along multiple planes.

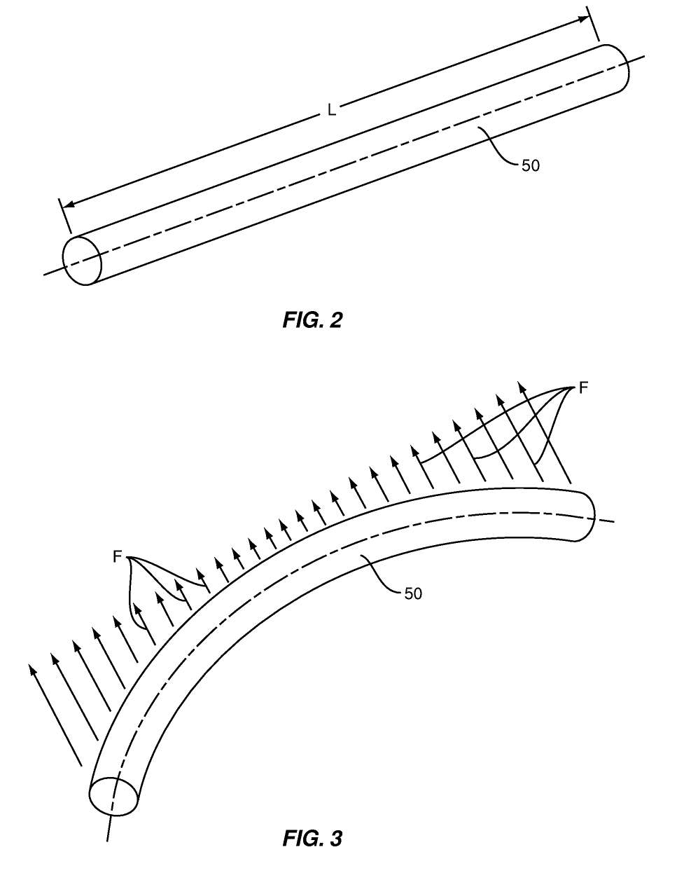

One embodiment of treating the spinal deformity utilizes a flexible member with elastic properties that impose a corrective force on the vertebral members 90. FIG. 2 illustrates one embodiment of a member 50 that includes an elongated rod. Members 50 may include a variety of configurations including rods and plates. The length L of the member 50 may vary depending upon the length of the deformed spine. The length L may extend along the entire length of the deformity, or may extend a lesser distance than the entire deformity.

The member 50 may be constructed from a variety of flexible surgical grade materials. Exemplary materials for the member 50 include polyurethane, silicone, silicone-polyurethane, polyolefin rubbers, hydrogels, and the like. Other suitable materials may include nitinol or other pseudoelastic alloys. Further, combinations of pseudoelastic alloys and non-metal elastic materials may be suitable. The elastic materials may be resorbable, semi-resorbable, or non-resorbable. Other exemplary materials for the member 50 include polymers such as polyetheretherketone (PEEK), polyethylene terephthalate (PET), polyester, polyetherketoneketone (PEKK), polyacetic acid materials (including polyactide and poly-DL-lactide), polyaryletherketone (PAEK), carbon-reinforced PEEK, polysulfone, polyetherimide, polyimide, and ultra-high molecular weight polyethelene (UHMWPE), and cross-linked UHMWPE, among others. Metals or ceramics can also be used, such as cobalt-chromium alloys, titanium alloys, nickel titanium alloys, memory wire, stainless steel alloys, calcium phosphate, alumina, pyrolytic carbon, and carbon fibers. Combinations of these materials, including combinations of metals and non-metals, are also contemplated.

The member 50 assumes a first, non-stressed orientation when no external forces are acting upon it. In the embodiment illustrated in FIG. 2, member 50 is substantially straight in the first, non-stressed orientation. An external force may be applied to deform the member to a second orientation. FIG. 3 illustrates the member 50 deformed to a second, curved orientation. The deformation to the second orientation imparts a stress to the member 50. The elastic properties of the member 50 induce a force F that acts to straighten the member 50 back towards the first, unstressed orientation. This force F acts to treat the spinal deformity when the member 50 is attached to the vertebral members 90.

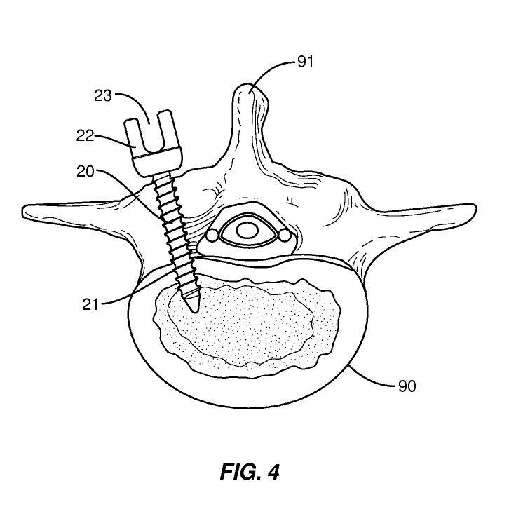

FIGS. 4-8 illustrate the steps of one method of inserting and attaching the member 50 within a patient. Anchors 20 are initially attached to the vertebral members 90, such as within the pedicles as illustrated in FIG. 4. The anchors 20 include a shaft 21 that extends into the vertebral member 90, and a head 22 positioned on the exterior. Head 22 may be fixedly connected to the shaft 21, or provide movement in one or more planes. Head 22 further includes a receiver 23 to receive the member 50. A set screw (not illustrated) is sized to engage with the head 22 to capture the member 50 within the receiver 23.

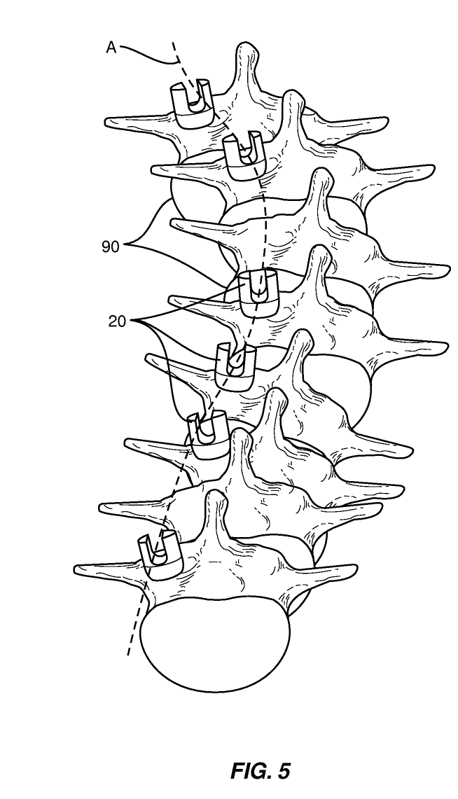

FIG. 5 schematically illustrates the vertebral members 90 that form the deformed spine. An anchor 20 is mounted to vertebral members 90 along a section of the spine. An anchor 20 may be placed within each vertebral member 90 along the deformed spine, or within selected vertebral members 90 as illustrated in FIG. 5. The anchors 20 are arranged to form a row A. In one embodiment, each anchor 20 is positioned at substantially the same lateral position within the respective vertebral member 90.

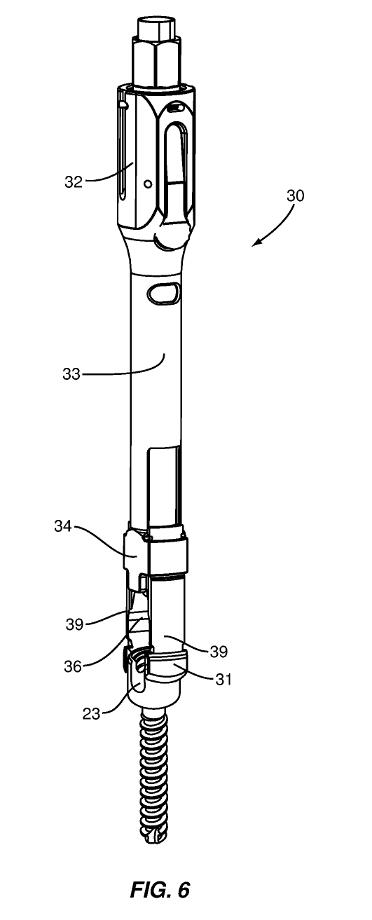

In one embodiment as illustrated in FIG. 6, an extender 30 may be connected to one or more of the anchors 20. The extender 30 includes a tubular element 33 with a distal end 31 and a proximal end 32. The tubular element 33 includes a length such that the proximal end 32 extends outward from the patient when the distal end 31 is mounted to the anchor 20. The distal end 31 includes a pair of opposing legs 39 that connect to the head of the anchor 20. The legs 39 form an opening that aligns with the receiver 23 to form a window 36. A sliding member 34 is movably positioned on the exterior of the tubular element 33 and located in proximity to the distal end 31. The sliding element 34 is axially movable along the tubular element to adjust a size of the window 36. One example of an extender 30 is the Sextant Perc Trauma Extender available from Medtronic Sofamor Danek of Memphis, Tenn.

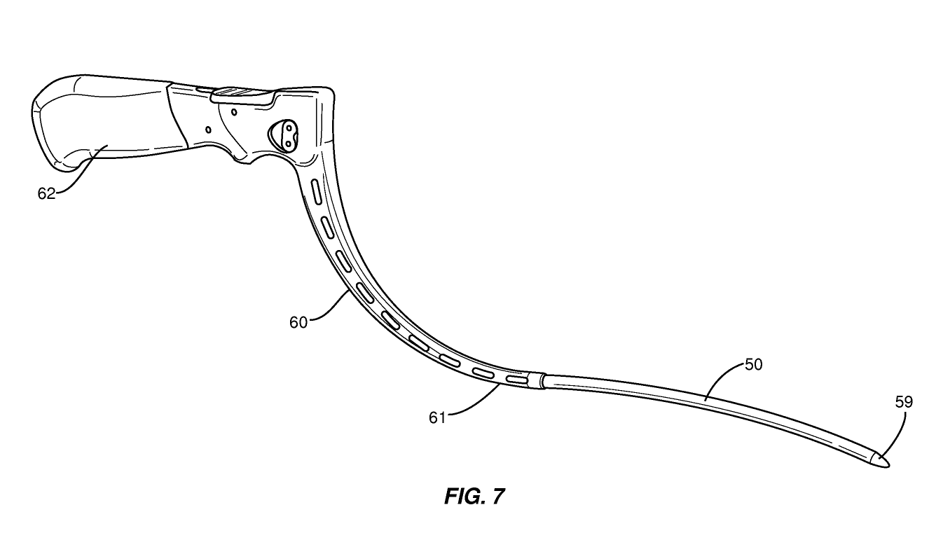

As illustrated in FIG. 7, the member 50 may be attached to an inserter 60 for insertion into the patient. Inserter 60 includes a handle 62 with an elongated neck 61. The distal end of the neck 61 is configured to receive the member 50. The member 50 may be curved as illustrated in FIG. 7 to facilitate insertion into the patient. The curved shape may also apply additional forces to particular lengths of the spine when the member 50 is rotated. In another embodiment, member 50 is substantially straight prior to insertion into the patient.

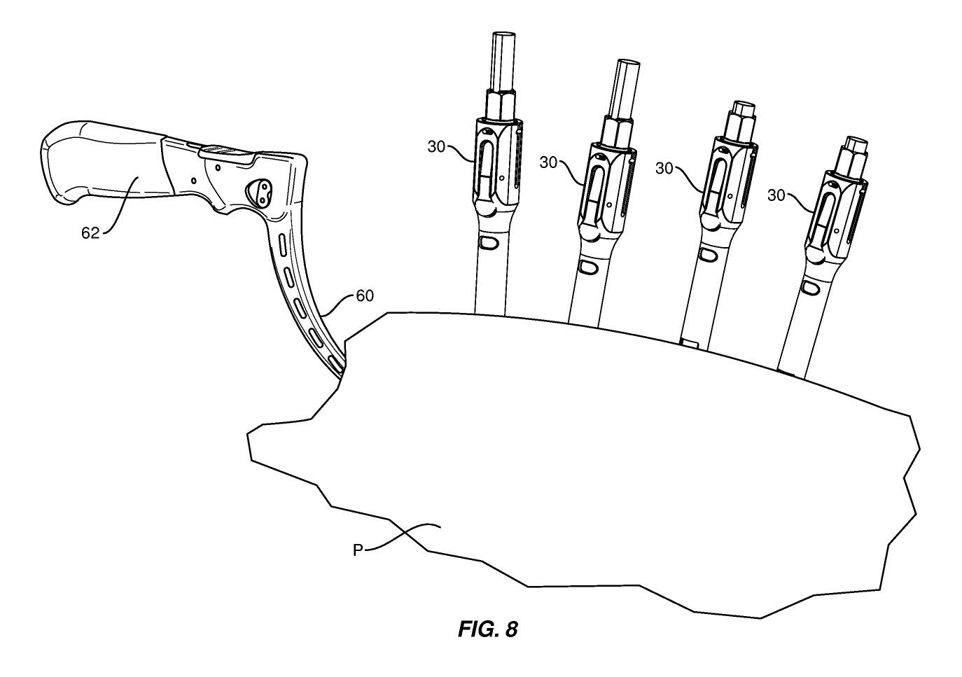

FIG. 8 illustrates one embodiment of the inserter 60 percutaneously inserting the member 50 into the patient P. After the member 50 is attached to the inserter 60, the distal end 59 of the member 50 is initially moved into an incision in the patient. The distal end 59 is then moved into the patient and through the first window 36 formed by the first anchor 20 and first extender 30. The movement of the member 50 is continued with the distal end 59 being moved through the remaining windows 36 formed by the extenders 30 and anchors 20. As illustrated in FIG. 8, the insertion process is performed percutaneously by the surgeon manipulating the handle 62 of the inserter 60 which remains on the exterior of the patient P. In one embodiment, movement of the member 50 through the patient P is performed using fluoroscopy imaging techniques.

Because the spine is deformed and the anchors 20 are positioned in a curved row A as illustrated in FIG. 5, the member 50 is deformed as it is inserted through the extenders 30. The flexibility of the member 50 allows for the bending as it is being moved through the extenders 30. Once the member 50 is in position through each of the extenders 30, set screws engage with the heads 22 of the anchors 20 to capture the member 50 within the receivers 23.

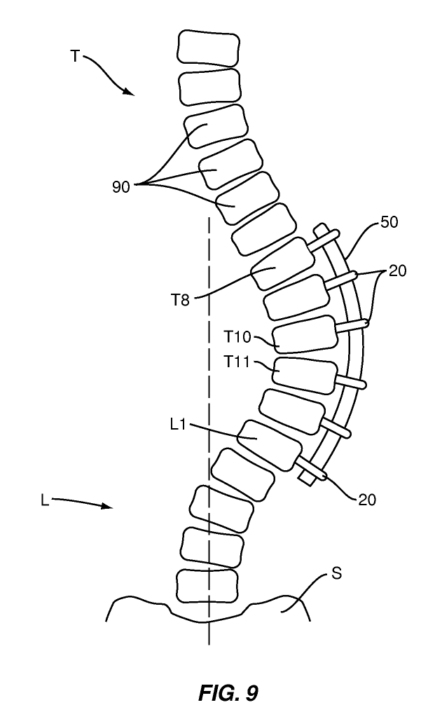

FIG. 9 illustrates the member 50 attached to the vertebral members 90 through the anchors 20. The elastic properties of the member 50 induce a force that tends to straighten the member 50 back towards its unstressed orientation. As the member 50 is urged to return to a straightened orientation, the member 50 imparts a corrective force on the vertebral members 90. In this embodiment, the corrective force may not immediately realign the vertebral members 90 after attachment of the member 50. The elastic nature of the member 50 instead induces a continuous corrective force on the vertebral members 90. Because of this continuous corrective force, the movement of the vertebral members 90 to the corrected position may occur gradually over time.

In one embodiment as illustrated in FIGS. 7, the member 50 is curved, or otherwise bent in the first, unstressed orientation. This shape, referred to as a pre-bent shape, may be established to apply specific corrective forces to the individual vertebral members 90. In one embodiment, the shape of the corrective member 50 is determined by studying the flexibility of the spinal deformity prior to the procedure. The shape of the member 50 corresponds to the needed displacement to translate and/or rotate the vertebral members 90 into alignment. Member 50 may be bent in one, two, or three dimensions depending on the amount of correction needed for the vertebral members 90 in the coronal, sagittal, and axial planes.

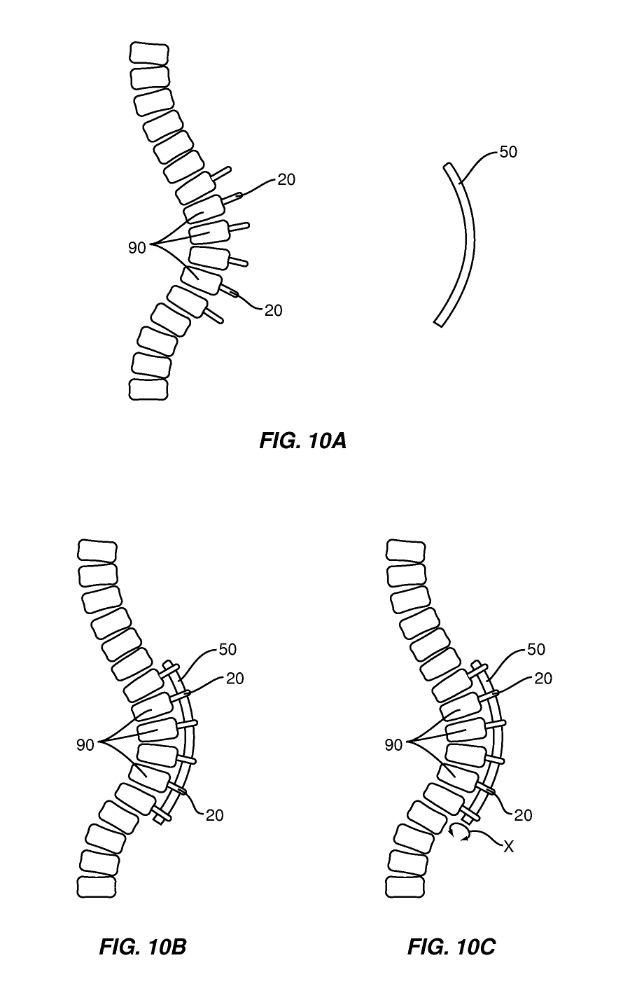

In one embodiment using a pre-bent shape, the member 50 is inserted into the patient in a first position relative to the vertebral members 90, and is then rotated to a second position. FIGS. 10A-10C schematically illustrate one method using a pre-bent member 50. FIG. 10A illustrates the vertebral members 90 in a deformed shape. Member 50 is pre-bent, and in this embodiment, the shape roughly matches the shape of the deformed spine. As illustrated in FIG. 10B, the pre-bent shape of the member 50 facilitates insertion and positioning the member 50 within the anchors 20 attached to the vertebral members 90. In this embodiment, the member 50 is inserted into the patient and moved through each of the windows 36 formed between the extenders 30 and anchors 20. In one embodiment, set screws may be loosely connected to the anchors 20 to prevent the member 50 from escaping during rotation.

Once the member 50 extends through the anchors 20, the member 50 is rotated as illustrated by arrow X in FIG. 10C. Rotation causes the member 50 to become deformed from the original pre-bent orientation to a second, stressed orientation. Once rotated, the member 50 is fixedly attached to the anchors 20, such as by set screws that engage the heads 22 to capture the member 50 within the receivers 23. The amount of rotation may vary depending upon the shape of the deformed spine, and the shape of the member 50. The rotation may cause the member to move from a first initial plane, into a second plane. This movement applies a corrective force to the vertebral members 90. The amount of rotation to move between the planes may vary. In one embodiment, the rotation may vary from between about 10° to about 180°. In one embodiment with a pre-bent member 50 in a substantially C-shape, the member 50 is rotated about 180°.

In one embodiment the member 50 is constructed of a shape memory alloy (SMA). The member 50 may be cooled to below body temperature, then bent to a first orientation, or placed under stress, to approximate the curvature of the deformed spine. The member 50 of this embodiment will not exert a force to return towards its first, unstressed orientation while still at the lower temperature. The member 50 is then inserted into the patient and attached to the anchors 20. As the member 50 warms to body temperature, the stress is released and the member 50 tends to move towards an unstressed second orientation thereby imparting a corrective force on the vertebral members 90. The movement of the vertebral members to the corrected position may occur gradually over a period of time.

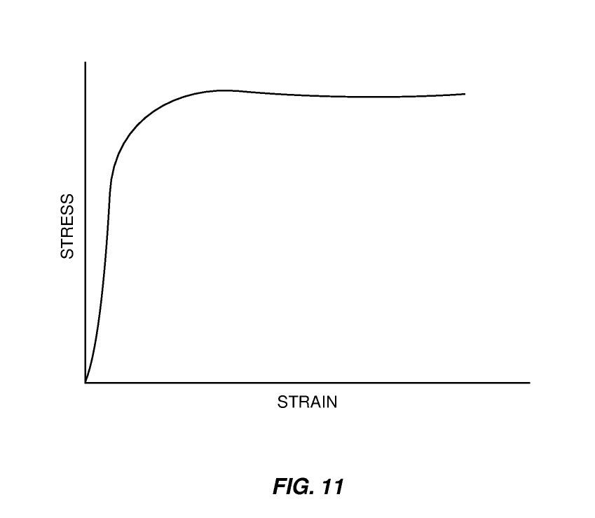

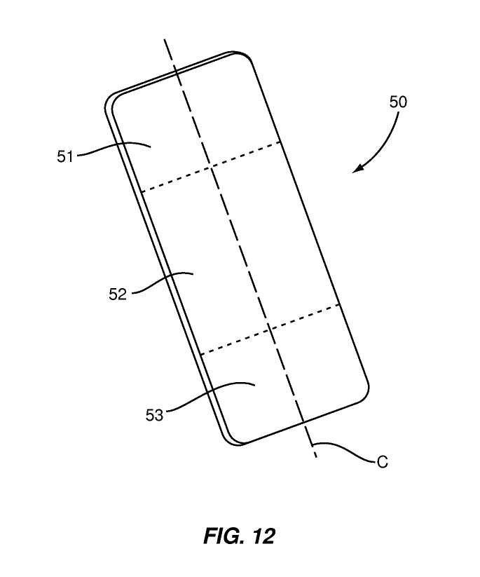

In one embodiment using a material with elastic memory, the member is constructed of polyetheretherketone (PEEK). The stress-strain curve for PEEK is relatively flat as shown in FIG. 11. This physical characteristic is beneficial because the member 50 undergoes different amounts of bending, or strain, along its length. In one embodiment illustrated in FIG. 9 with a member 50 that is substantially straight in an unstressed orientation, a central portion of the member 50 undergoes a smaller amount of bending than an end of the member 50. Since the stress-strain curve is relatively flat, a more uniform force is applied to each of the vertebral members 90 to which the member 50 is attached. Thus, the force applied to the vertebral member T11 is similar to the force applied to vertebral member L1.

In one embodiment, the member 50 is constructed to include different flexibilities along the length. FIG. 12 schematically illustrates one embodiment with three separate sections 51, 52, 53 extending along the length. Each of the sections 51, 52, 53 includes a different flexural rigidity that differs from that of an adjoining section. The member 50 may be constructed to correspond to the specific nature of the spinal deformity. Using the member 50 of FIG. 12 with the deformity illustrated in FIG. 1, a central section 52 may be constructed of a material with a higher flexural rigidity than end sections 51, 53. Positioning the member 50 such that the central section 52 is adjacent to vertebral member T10 may impart a greater corrective force to vertebral member T10 without over correcting vertebral member T8. The number of different sections within the member 50 may vary depending upon the context of use.

Member 50 may also include different cross-sectional shapes and sizes to vary the flexural rigidity of the member 50 along its length to impart a variety of corrective forces on the vertebral members 90. By varying the cross-sectional area, the flexural rigidity may also be varied, allowing the member 50 to be constructed to more accurately apply a desired corrective force to individual vertebral members 90 or groups of vertebral members 90. A variety of shapes may be considered depending upon the context of use, and desired corrective forces. Examples of various cross-sectional shapes and sizes are disclosed in U.S. patent application Ser. No. 11/342,195 entitled “Spinal Rods Having Different Flexural Rigidities about Different Axes and Methods of Use”, filed on Jan. 27, 2006, hereby incorporated by reference.

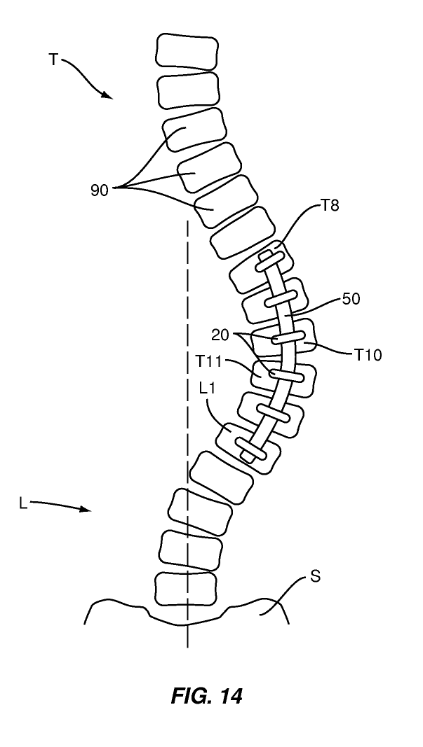

In some embodiments as illustrated in FIGS. 9, 10A-C, and 14, a single member 50 is attached to the vertebral members 90. The members 50 may be attached at a variety of different positions. FIGS. 9 and 10A-C illustrate the member 50 attached to a lateral side of the vertebral members 90. FIG. 14 illustrates the member 50 attached to a posterior side of the vertebral members 90. It is understood that the member 50 may be located at various other positions along the vertebral members 90. In one embodiment, member 50 extends along a portion of a lateral side and an anterior side of the vertebral members 90.

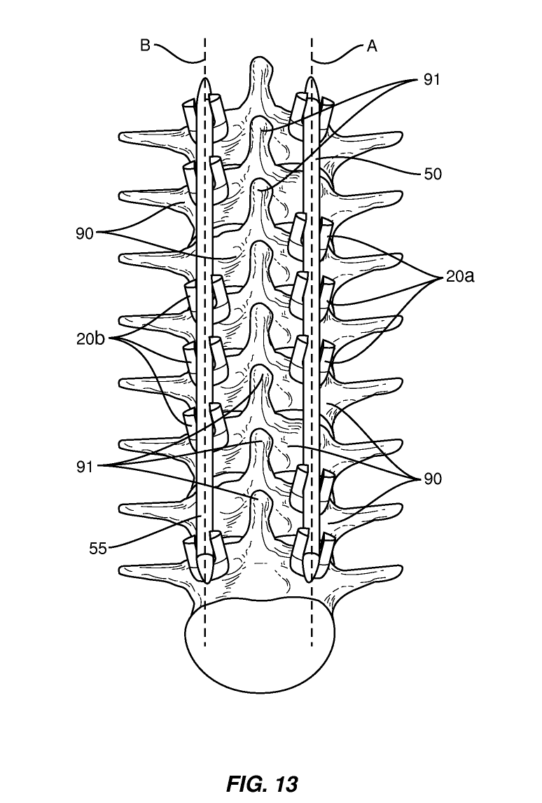

As previously discussed, vertebral members 90 may be misaligned both laterally and rotationally. Vertebral members 90 may also be misaligned in more than one plane. A single member 50 attached to the spine may provide corrective forces for only a limited number of misalignments when a variety of misalignments are present simultaneously. As illustrated in FIG. 13, a second member 55 may be used to apply different corrective forces to the vertebral members 90. The second member 55 may apply corrective forces to the same vertebral members 90 as the first member 50, or to different vertebral members 90. The second member 55 may also differ in flexural rigidity from the first member 50. FIG. 13 illustrates the first member 50 aligned along a lateral position A on one side of the spinous process 91, and the second member 55 aligned along lateral position B on an opposite side of the spinous process 91. Alternately, the second member 55 may be aligned along the same side of the spinous process 91 as the first member 50 (not shown).

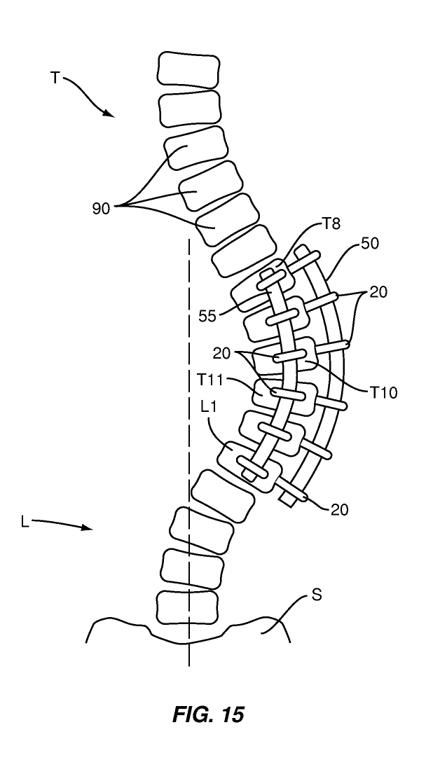

FIG. 15 illustrates another embodiment with the first member 50 positioned along a lateral position of the vertebral members 90, and second member 55 positioned along a posterior side of the vertebral members 90.

As used herein, the term “elastic” means the ability of a material to deform in response to an applied external stress and to return essentially to an initial form once the external stress is removed.

In one embodiment, the devices and methods are configured to reposition and/or realign the vertebral members 90 along one or more spatial planes toward their normal physiological position and orientation. The spinal deformity is reduced systematically in all three spatial planes of the spine, thereby tending to reduce surgical times and provide improved results. In one embodiment, the devices and methods provide three-dimensional reduction of a spinal deformity via a posterior surgical approach. However, it should be understood that other surgical approaches may be used, including, a lateral approach, an anterior approach, a posterolateral approach, an anterolateral approach, or any other surgical approach.

The anchors 20 described above are some embodiments that may be used in the present application. Other examples include spinal hooks configured for engagement about a portion of a vertebral member 90, bolts, pins, nails, clamps, staples and/or other types of bone anchor devices capable of being anchored in or to the vertebral member 90. In one embodiment, anchors 20 include fixed angle screws.

In still other embodiments, anchors 20 may allow the head portion 22 to be selectively pivoted or rotated relative to the threaded shaft portion 21 along multiple planes or about multiple axes. In one such embodiment, the head portion 22 includes a receptacle for receiving a spherical-shaped portion of a threaded shaft therein to allow the head portion 22 to pivot or rotate relative to the threaded shaft portion. A locking member or crown may be compressed against the spherical-shaped portion via a set screw or another type of fastener to lock the head portion 22 at a select angular orientation relative to the threaded shaft portion. The use of multi-axial anchors 20 may be beneficial for use in the lower lumbar region of the spinal, and particularly below the L4 vertebral member, where lordotic angles tend to be relatively high compared to other regions of the spinal column. Alternatively, in regions of the spine exhibiting relatively high intervertebral angles, the anchors 20 may include a fixed angle.

The present embodiments may be used to treat a wide range of spinal deformities. The devices and methods may be used to treat spinal deformities including scoliosis, kyphotic deformities such as Scheurmann's kyphosis, fractures, congenital abnormalities, degenerative deformities, metabolic deformities, deformities caused by tumors, infections, trauma, and other abnormal spinal curvatures.

In one embodiment, the treatment of the deformity is performed percutaneously. In other embodiments, the treatment is performed with an open approach, semi-open approach, or a muscle-splitting approach.

Spatially relative terms such as “under”, “below”, “lower”, “over”, “upper”, and the like, are used for ease of description to explain the positioning of one element relative to a second element. These terms are intended to encompass different orientations of the device in addition to different orientations than those depicted in the figures. Further, terms such as “first”, “second”, and the like, are also used to describe various elements, regions, sections, etc and are also not intended to be limiting. Like terms refer to like elements throughout the description.

As used herein, the terms “having”, “containing”, “including”, “comprising” and the like are open ended terms that indicate the presence of stated elements or features, but do not preclude additional elements or features. The articles “a”, “an” and “the” are intended to include the plural as well as the singular, unless the context clearly indicates otherwise.

The present invention may be carried out in other specific ways than those herein set forth without departing from the scope and essential characteristics of the invention. The corrective member may be inserted in a top-to-bottom direction or a bottom-to-top direction. The present embodiments are, therefore, to be considered in all respects as illustrative and not restrictive, and all changes coming within the meaning and equivalency range of the appended claims are intended to be embraced therein.

What is claimed is:

1. A method for fusionless correction of a spinal deformity, comprising:
contouring a member constructed of elastic material along a deformed spine;
positioning the member along the deformed spine using anchors secured in adjacent vertebral members;
adjusting the member in the anchors whereby corrective forces are applied through the anchors to the deformed spine; and
securing the member in the anchors.

2. The method of claim 1, wherein the anchors are pedicle screws.

3. The method of claim 1, wherein the member is originally in a straight orientation prior to insertion and then bent to match the curvature of the deformed spine.

4. The method of claim 2, wherein the member is pre-bent to match the curvature of the deformed spine before anchoring in the pedicle screws.

5. The method of claim 4, wherein the pre-bent member is rotated about its longitudinal axis in the pedicle screws.

6. The method of claim 5, wherein the pre-bent member is rotated about 180° about its longitudinal axis.

7. The method of claim 5, wherein the pre-bent member is shifted along its longitudinal axis for alignment along the deformed spine.

8. The method of claim 2, wherein the member is made of shape-memory alloy (SMA).

9. The method of claim 2, wherein the anchors include multi-axial screws.

10. The method of claim 1, wherein the member is constructed from a group of metals comprising stainless steel, titanium, nitinol, and cobalt-chrome.

11. The method of claim 1, wherein the member is constructed from a polymeric material.

12. The method of claim 1, wherein the member is a composite constructed from a group of materials comprising polymers, ceramics, and metals.

13. The method of claim 4, wherein the member is pre-bent to match the curvature of the deformed spine in the frontal plane and facilitate anchoring of the member in the pedicle screws.

14. The method of claim 2, wherein the member is inserted into the pedicle screws in a percutaneous technique.

15. A device for correcting a spinal deformity, comprising:
at least one pre-stressed elastic spinal member;
anchors mounted along the pedicles of the spine and adapted to receive the spinal member;
wherein the anchors provide both longitudinal and rotational movement of the spinal member whereupon securing the spinal member to the anchors provides a constant or substantially constant correction force to the spine and maintains the constant or substantially constant correction force until the spinal deformities are fully or substantially fully corrected.

16. The device according to claim 15, wherein the pre-stressed spinal member is straight and then bent to match the spinal curve.

17. The device according to claim 15, wherein the pre-stressed spinal member is pre-bent to match spinal deformities in the frontal plane and rotated about 180° about its axis while located in the anchors.

18. The device according to claim 15, wherein the pre-stressed spinal member is made of SMA.

19. The device according to claim 15, wherein the anchors are pedicle screws.

20. The device according to claim 15, wherein the pre-stressed elastic spinal member is constructed from a group of metals comprising stainless steel, titanium, nitinol, and cobalt chrome.

21. A device for correcting a spinal deformity, comprising:
an elastic spinal member with a first, non-stressed orientation;
anchors mounted along the pedicles of the spine and adapted to receive the spinal member;
securing the spinal member to the anchors places the spinal member in a second, stressed orientation to provide a constant or substantially constant correction force to the spine and maintain the constant or substantially constant correction force to treat the spinal deformity.

22. A system for correcting spinal deformities without using fusion, said system comprising;
at least one pre-stressed spinal member constructed of elastic material; and
a plurality of anchors secured in adjacent vertebrae that allows both longitudinal and axial rotational movement of the spinal member before fixedly securing said member.

23. The system of claim 22, wherein the pre-stressed spinal member is made of SMA.

24. The system of claim 22, wherein the pres-stressed spinal member is metal and pre-bent and is rotated about 180° about its axis when located in the anchors.
